(19) United States
(12) Patent Application Publication
(10) Pub. No.: US 2022/0280727 A1
(43) Pub. Date: Sep. 8, 2022

(54) INJECTION DEVICE

(71) Applicant: Owen Mumford Limited, Woodstock, Oxfordshire (GB)

(72) Inventor: Toby COWE, Woodstock, Oxfordshire (GB)

(21) Appl. No.: 17/608,869

(22) PCT Filed: May 21, 2020
(86) PCT No.: PCT/EP2020/064222

§ 371 Date: Nov. 4, 2021

(30) Foreign Application Priority Data
May 6, 2019 (GB) 1906941.8

(51) Int. Cl. A61M 5/315; A61M 5/20

(52) U.S. Cl. CPC A61M 5/31541; A61M 5/31553; A61M 5/31585; A61M 5/2033; A61M 5/31593; A61M 2207/00

(57) ABSTRACT

An injection device for delivering a dose from a cartridge or syringe. The injection device comprises a housing, a drive shaft, a drive spring for urging the drive shaft to rotate relative to the housing, and a plunger coupled to the drive shaft such that rotation of the drive shaft relative to the housing causes the plunger to move axially relative to the housing. The device further comprises a dose setting member moveable relative to the drive member between a first position in which the dose setting member is rotatable relative to the housing for setting a dose and a second position in which the dose setting member is rotationally fixed relative to the housing for delivering a dose, and a stop member. The stop member is engaged between the dose setting member and the drive shaft such that relative rotation between the dose setting member and the drive shaft during dose setting causes the stop member to travel relative to a first stop surface, and rotation of the dose setting member together with the drive shaft during dose delivery does not cause the first stop member to travel relative to the first stop surface. If the stop member abuts the first stop surface during relative rotation between the dose setting member and the drive shaft, continued travel of the stop member and hence continued relative rotation between the dose setting member and the drive shaft is prevented.

### TECHNICAL FIELD

The present invention relates to an injection device for delivering doses from a cartridge or a syringe.

### BACKGROUND

Injection devices can be used to deliver a medicament (e.g. insulin) from a cartridge or syringe. Some types of injection devices allow multiple doses to be delivered from the cartridge or syringe and allow the user to set the volume for each dose.

WO 2011/045611 discloses an injection device comprising a dose setting arrangement that allows a user to set a dose to be delivered from a cartridge or syringe, and a drive mechanism comprising a drive spring and a clutch arrangement, wherein the clutch arrangement is operable during a dose setting routine to isolate the dose setting arrangement from the force of the drive spring. In this way, the user can set a dose without having to work against the force of the drive spring.

It is desirable to provide a mechanism that prevents the user from setting a dose that exceeds the total deliverable volume of substance remaining in the cartridge. In particular, it is desirable to provide such a mechanism in the type of device disclosed in WO 2011/045611. However, due to the specific configuration and interactions between the components of such a device, it is not straightforward to implement such a mechanism.

### STATEMENTS OF INVENTION

According to an aspect of the present invention, there is provided an injection device for delivering a dose from a cartridge or syringe, the injection device comprising:
a housing for receiving the cartridge or syringe;
a drive shaft rotatably mounted within the housing;
a drive spring configured to directly or indirectly urge the drive shaft to rotate relative to the housing;
a plunger coupled to the drive shaft such that rotation of the drive shaft relative to the housing directly or indirectly causes the plunger to move axially relative to the housing for delivering a dose from the cartridge or syringe, in use;
a dose setting member moveable relative to the drive shaft between a first position for setting a dose and a second position for delivering a dose;
wherein when the dose setting member is in its first position, the drive shaft is rotationally fixed relative to the housing and the dose setting member is rotatable relative to the drive shaft to set the dose; and
wherein when the dose setting member is in its second position, the drive shaft is permitted to rotate relative to the housing under the influence of the drive spring and the dose setting member is rotationally fixed relative to the drive shaft such that the rotation of the drive spring relative to the housing causes the dose setting member to rotate together with the drive shaft relative to the housing;
the injection device further comprising:
a stop member engaged between the dose setting member and the drive shaft such that relative rotation between the dose setting member and the drive shaft causes the stop member to travel relative to a first stop surface, and rotation of the dose setting member together with the drive shaft does not cause the first stop member to travel relative to the first stop surface;
wherein if the stop member abuts the first stop surface during relative rotation between the dose setting member and the drive shaft, continued travel of the stop member and hence continued relative rotation between the dose setting member and the drive shaft is prevented.

Rotation of the dose setting member relative to the drive shaft in a direction to increase the set dose can cause the stop member to travel toward the first stop surface. Conversely, rotation of the dose setting member relative to the drive shaft in a direction to decrease the set dose can cause the stop member to travel away from the first stop surface.

The position of the stop member relative to the first stop surface (at any point when the injection device is at rest) can correspond to the total deliverable volume of substance (remaining) in the cartridge or syringe.

The stop member can be threadedly engaged with a first helical thread on one of the dose setting member and the drive shaft, and the stop member can be rotationally fixed and axially movable relative to the other of the dose setting member and the drive shaft, such that relative rotation between the dose setting member and the drive shaft causes the stop member to move along the first helical thread.

The first stop surface can be located on the first helical thread. Alternatively, the first stop surface can be located on the other of the dose setting member and the drive shaft.

The first helical thread can be located on the drive shaft and the stop member can be rotationally fixed and axially movable relative to the dose setting member.

The drive shaft can comprise formations and the dose setting member can comprise corresponding formations that are engaged to prevent relative rotation between the drive shaft and the dose setting member when the dose setting member is in its second position, and are not engaged when the dose setting member is in its first position to permit relative rotation between the dose setting member and the drive shaft. The formations on the drive shaft and corresponding formation on the dose setting member can be in the form of axially extending splines that are interlocked in the circumferential direction to prevent relative rotation between the drive shaft and the dose setting member when the dose setting member is in its second position, and are not interlocked when the dose setting member is in its second position.

The dose setting member can be axially moveable relative to the drive shaft between its first position and its second position.

The injection device can further comprise a clutch member coupled to the dose setting member and moveable by the dose setting member between an engaged position in which the clutch member is engaged between the drive shaft and the housing to prevent relative rotation between the drive shaft and the housing, and a disengaged position in which the clutch member is not engaged between the drive shaft and the housing such that relative rotation between the drive shaft and the housing is permitted; wherein the dose setting member and the clutch member are coupled such that when the dose setting member is moved between its first position and its second position, the clutch member is moved between its engaged position and its disengaged position respectively.

The clutch member can comprise formations that engage with corresponding formations on the housing and corresponding formations on the drive shaft to prevent relative rotation between the drive shaft and the housing when the clutch member is in its engaged position, and that move out of engagement with the corresponding formations on either or both of the housing and the drive shaft when the clutch member is moved to its disengaged position. For example, the formations on the clutch member, the corresponding formations on the housing and the corresponding formations on the drive shaft can be in the form of axially extending splines that interlock in the circumferential direction when the clutch member is in its engaged position.

The clutch member can be axially moveable relative to the housing between its engaged position and its disengaged position. The dose setting member can be coupled to the clutch member such that axial movement of the dose setting member axially moves the clutch member.

The dose setting member can be rotatable relative to the clutch member (in at least the first position of the dose setting member or both the first position and the second position of the dose setting member).

The injection device can further comprise a dose button coupled to the dose setting member, wherein the dose button is rotatable relative to the housing and movable relative to the housing between a first position and a second position, and the dose button and the dose setting member are coupled such that:
when the dose button is in its first position, the dose setting member is in its first position and the dose setting member is rotationally fixed relative to the dose button;
when the dose button is in its second position, relative rotation between the dose setting member and the dose button is permitted; and
when the dose button is moved between its first position and its second position, the dose setting member is moved between its first position and second position respectively.

Thus, the dose button can provide the user with indirect control over the dose setting member. The dose setting member can be arranged internally within the housing, whereas at least a portion of the dose button can be arranged externally of the housing (e.g. protruding from one axial end of the housing). To set a dose, the user can rotate the dose button relative to the housing, which rotates the dose setting member relative to the housing. To deliver a dose, the user can move the dose button from its first position to its second position, which moves the dose setting member from its first position to its second position. When the dose setting member is rotating relative to the housing during dose delivery, the button will not rotate, which helps to prevent the user from interfering with the dose delivery operation.

The dose button can comprise formations and the dose setting member can comprise corresponding formations that engage to prevent relative rotation between the dose button and the dose setting member when the dose button is in its first position and move out of engagement to permit relative rotation between the dose button and the dose setting member when the dose button is moved from its first position to its second position. For example, the formations of the dose button and the corresponding formations on the dose setting member can be in the form of axially extending splines that interlock in the circumferential direction when the dose button is in its first position and move out of interlocking engagement when the dose button is moves to its second position.

The injection device can further comprise a return spring configured to bias the dose button toward its first position.

The dose button can be axially moveable between its first position and its second position. The dose button and the dose setting member can be coupled such that axial movement of the dose button axially moves the dose setting member.

The axial displacement of the plunger during dose delivery (and hence the dose delivered) can be determined by the rotational displacement of the drive shaft during dose delivery. The dose setting member can set the dose by defining the maximum rotational displacement of the drive shaft during dose delivery.

For example, the injection device can further comprise a control member engaged between the housing and the dose setting member such that relative rotation between the housing and the dose setting member causes the control member to travel relative to a second stop surface; wherein:
during dose delivery, relative rotation between the dose setting member and the housing under the influence of the drive spring causes the control member to move toward the second stop surface until the control member abuts the second stop surface, whereby continued rotation of the dose setting member relative to the housing is prevented; and
during dose setting, relative rotation between the dose setting member and the housing sets the dose by defining the position of the control member relative to the second stop surface.

Rotation of the dose setting member relative to the housing in one direction can cause the control member to travel away from the second stop surface (which corresponds to increasing the set dose). Rotation of the dose setting member relative to the housing in the other direction can cause the control member to travel toward the second stop surface (which corresponds to decreasing the set dose).

The control member can be threadedly engaged with a helical thread on the housing, and can be rotationally fixed and axially movable relative to the dose setting member.

The control member can provide a visual indication of the set dose, e.g. by numbers on the surface of the control member. The housing can include a window (e.g. an aperture or a transparent portion) that allows the user to view a number on the control member. When the control member is abutting the second stop surface, the control member can indicate to the user that the set dose is zero.

A third stop surface can be provided (e.g. on the housing) that limits the maximum distance that the control member can travel away from the second stop surface. The third stop surface can therefore limit the maximum dose that can be set for a single dose delivery.

The drive spring can be a torsion spring. The torsion spring can be engaged between the housing and the drive shaft for applying a torque between the housing and the drive shaft. One end of the torsion spring can be attached to the housing and the other end of the torsion spring can be attached to the drive shaft.

Alternatively, the drive spring can be a compression spring. The compression spring can be axially engaged between the plunger and the drive shaft for applying a linear force (in the axial direction) between the plunger and the drive shaft. One end of the drive spring can be engaged with a forward-facing surface on the drive shaft, and the other end of the drive spring can be engaged with a rearward-facing surface on the plunger. The compression spring can also apply a torque between the plunger and the drive shaft in addition to the linear force, e.g. by torsioning the compression spring during assembly of the injection device.

Alternatively, the drive spring can be a power spring. A power spring is a typically in the form of a flat metal band wound around a central arbor (or shaft). The power spring can be engaged between the housing and the drive shaft for applying a torque between the housing and the drive shaft. The power spring can comprise an outer end attached to the housing and an inner end attached to an arbor, wherein the arbor is integrally formed with or attached to the drive shaft. The arbor can be rotationally fixed relative to the drive shaft.

Therefore, in some embodiments, one end of the drive spring can be coupled to the drive shaft, and the other end of the drive spring can be coupled to the housing, and in other embodiments, one end of the drive spring can be coupled to the drive shaft, and the other end of the drive spring can be coupled to the plunger.

When the dose setting member is in its first position, the dose setting member can be completely isolated from the force of the drive spring. In other words, there is no torque or force transmission path from the drive spring to the dose setting member.

During assembly of the injection device, the drive spring can be preloaded such that prior to a first dose setting operation, the drive spring has enough energy to supply the force required to perform at least one dose delivery, preferably a plurality of dose deliveries, without any input of energy by the user into the drive spring.

During assembly of the injection device, the drive spring can preloaded such that prior to a first dose setting operation, the drive spring has enough energy to supply the force required to deliver the total deliverable volume of substance in the cartridge or syringe over one or more dose deliveries without the user needing to input any energy into the drive spring.

The drive shaft can comprise a first drive shaft portion and a separate second drive shaft portion, wherein:
the drive spring is configured to directly or indirectly urge the first drive shaft portion to rotate relative to the housing;
the first drive shaft portion is engaged with the second drive shaft portion such that the first drive shaft portion is rotationally fixed relative to the second drive shaft portion; and
the stop member is engaged between the dose setting member and the second drive shaft portion.

By providing this split drive shaft, assembly of the injection device can be easier because angular alignment between components (e.g. the second drive shaft portion, the stop member and the dose setting member) can be maintained during insertion into the housing without interference from the drive spring.

Optionally:
the clutch member is integrally formed with or is attached to the second drive shaft portion such that the clutch member is axially fixed relative to the second drive shaft portion;
the second drive shaft portion is axially movable relative to the first drive shaft portion; and
when the clutch member is in its engaged position, the clutch member is engaged between the first drive shaft portion and the housing to prevent relative rotation between the first drive shaft portion and the housing.

In another aspect of the invention, there is provided a method of assembling an injection device, the injection device comprising:
a drive shaft comprising a threaded portion;
a plunger comprising a corresponding threaded portion for engaging with the threaded portion of the drive shaft; and
a compression spring;
   the method comprising the steps of:
engaging a first end of the compression spring with the drive shaft;
engaging a second end of the compression spring with the plunger, such that the compression spring is engaged between the drive shaft and the plunger in the axial direction; and
rotating the plunger relative to the drive shaft to threadedly engage the plunger with the drive shaft and cause the plunger to move axially relative to the drive shaft, such that the compression spring is simultaneously compressed and torsioned between the plunger and the drive shaft.

The drive shaft can comprise a seat and the step of engaging the first end of the compression spring with the drive shaft can comprise engaging the first end of the compression spring with the seat of the drive shaft. The plunger can also comprise a seat and the step of engaging the second end of the compression spring with the plunger can comprise engaging the second end of the compression spring with the seat of the plunger.

When the plunger is rotated relative to the drive shaft, the engagement between the first end of the compression spring and the seat of the drive shaft can restrict or resist the first end of the compression spring from rotating relative to the drive shaft, and the engagement between the second end of the compression spring and the seat of the plunger can restrict or resist the second end of the compression spring from rotating relative to the plunger.

The seat of the drive shaft and the seat of the plunger can be in the form of a recess.

The first end of the compression spring can comprise an axially extending first end formation. The first end formation can be received in the seat of the drive shaft during the step of engaging the first end of the compression spring with the drive shaft. The second end of the compression spring can comprise an axially extending second end formation. The second end formation can be received in the seat of the plunger during the step of engaging the second end of the compression spring with the plunger.

The drive shaft can comprise a plurality of seats arranged circumferentially about its longitudinal axis. The step of engaging the first end of the compression spring with the drive shaft can comprise engaging the first end of the compression spring with one of the plurality of seats of the drive shaft. The plunger can also comprise a plurality of seats arranged circumferentially about its longitudinal axis. The step of engaging the second end of the compression spring with the plunger can comprise engaging the second end of the compression spring with one of the plurality of seats of the plunger.

The plurality of seats of the drive shaft can be separated in the circumferential direction by circumferential walls. Each of the circumferential walls can be shaped such that the first end of the compression spring can be diverted toward one of the plurality of seats of the drive shaft during the step of engaging the first end of the compression spring with the drive shaft. The plurality of seats of the plunger can also be separated in the circumferential direction by circumferential walls. Each of the circumferential walls can be shaped such that the second end of the compression spring can be diverted toward one of the plurality of seats of the plunger during the step of engaging the second end of the compression spring with the plunger.

The drive shaft can comprise an axially extending bore. The method can further comprise inserting the compression spring into the bore of the drive shaft before engaging the first end of the compression spring with the drive shaft. The plunger can also comprise an axially extending bore. The method can further comprise receiving the compression spring into the bore of the plunger before engaging the second end of the compression spring with the plunger.

The injection device can further comprise a pin (a generally elongate member). The method can further comprise, before engaging the first end of the compression spring with the drive shaft:
inserting the pin through the drive shaft in an axial direction; and
arranging the compression spring over the pin such that the pin extends axially through the centre of the compression spring.

In another aspect of the invention, there is provided an injection device for delivering a dose from a cartridge or syringe, the injection device comprising:
a housing for receiving the cartridge or syringe;
a drive shaft rotatably mounted within the housing;
a plunger threadedly engaged with the drive shaft; and
a compression spring axially engaged between the drive shaft and the plunger and configured to urge the plunger to move axially relative to the drive shaft and the housing to deliver a dose from the cartridge or syringe, in use;
wherein the compression spring is in a compressed and torsioned state.

The compression spring can be in a compressed and torsioned state after assembly of the injection device and before first use of the injection device (i.e. before setting a first dose) by the user. The compression spring can indirectly urge (as opposed to directly urge) the drive shaft to rotate relative to the housing.

The drive shaft can comprise a seat in which a first end of the compression spring is engaged, and the plunger can comprise a seat in which a second end of the compression spring is engaged.

The seat of the drive shaft and the seat of the plunger can be in the form of a recess.

The first end of the compression spring can comprise an axially extending end formation engaged with the seat of the drive shaft. The second end of the compression spring can comprise an axially extending end formation engaged with the seat of the plunger.

The injection device can further comprise a pin extending axially through the compression spring. The outer surface of the pin can form an inner radial wall of the seat of the drive shaft and/or the plunger.

The drive shaft can comprise a plurality of seats arranged circumferentially about its longitudinal axis. The first end of the compression spring can be engaged with one of the plurality of seats. The plunger can comprise a plurality of seats arranged circumferentially about its longitudinal axis. The second end of the compression spring can be engaged with one of the plurality of seats.

The plurality of seats of the drive shaft can be separated in the circumferential direction by circumferential walls. Each of the circumferential walls can be shaped such that the first end of the compression spring can be diverted into one of the plurality of seats of the drive shaft when engaging the first end of the compression spring with the drive shaft during assembly of the injection device. The plurality of seats of the plunger can separated in the circumferential direction by circumferential walls. Each of the circumferential walls can shaped such that the second end of the compression spring can be diverted into one of the plurality of seats of the plunger when engaging the second end of the compression spring with the plunger during assembly of the injection device.

The drive shaft can comprise an axially extending bore. The plunger can be threadedly engaged with the drive shaft within the bore of the drive shaft. The compression spring can be located within the bore of the drive shaft. The plunger can also comprise an axially extending bore. The compression spring can be located within the bore of the drive shaft and the bore of the plunger.

The torque applied between the plunger and the drive shaft by the compression spring in its compressed and torsioned state can be less than the torque required to move the plunger to deliver the total deliverable volume of substance in the cartridge or syringe. The linear force applied by the compression spring in its compressed and torsioned state between the plunger and the drive shaft can be sufficient to move the plunger to deliver the total deliverable volume of substance in the cartridge or syringe.

### BRIEF DESCRIPTION OF THE DRAWINGS

The invention will now be described, by way of example only, with reference to the accompanying drawings, in which:

FIG. 1 shows a cross-section view of an injection device 100;

FIG. 2 shows a cross-section view of the injection device 100;

FIG. 3 shows a cross-section view of the injection device 100;

FIG. 4 shows a cross-section view of the injection device 100;

FIG. 5 shows a partial perspective view of the injection device 100;

FIG. 6 shows a partial perspective view of a component of the injection device 100;

FIG. 7 shows a perspective view of a component of the injection device 100;

FIG. 8 shows a cross-section view of an injection device 200;

FIG. 9 shows a perspective view of a component of the injection device 200;

FIG. 10 shows a cross-section view of the injection device 200;

FIG. 11 shows a cross-section view of the injection device 200;

FIG. 12 shows a cross-section view of an injection device 300;

FIG. 13 shows a cross-section view of an injection device 400;

FIG. 14 shows a cross-section view of the injection device 400;

FIG. 15 shows a perspective view of a component of the injection device 400;

FIG. 16 shows a perspective view of a component of the injection device 400;

FIG. 17 shows a partial perspective view of a component of the injection device 400.

### DETAILED DESCRIPTION

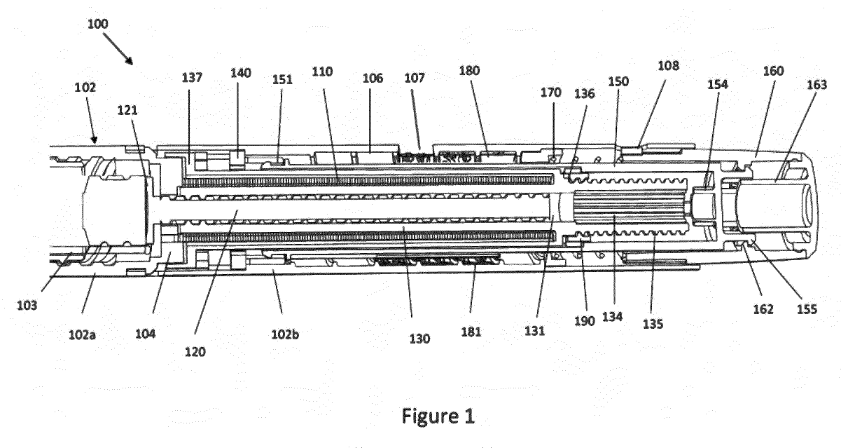

FIG. 1 shows a cross-sectional view of an injection device 100. The forward end of the device is the end of the device from which a dose is expelled and the forward direction is the direction in which a dose is expelled when the device is in use.

Injection device 100 comprises a housing 102 formed in two halves—a forward housing portion 102a and a rearward housing portion 102b—that are attached together, e.g. by a snap fit or glue, such that the two halves are rotationally and axially fixed relative to each other. The forward housing portion 102a defines a cavity 103 for receiving a cartridge or a syringe containing a substance (e.g. a medicament). The rearward housing portion 102b defines a cavity for receiving a drive mechanism for delivering the contents of the cartridge or syringe.

The drive mechanism comprises a drive spring 110, a plunger 120, a drive shaft 130, a clutch member 140, a dose setting member 150, a dose button 160, a return spring 170, a control member 180 and a stop member 190, all of which are co-axial with the longitudinal axis of the housing 102. The terms “axial”, “axially”, “in the axial direction” etc. used throughout this description refer to this longitudinal axis. The terms “outer” and “inner” are relative to this longitudinal axis in the radial direction.

The drive shaft 130 is in the form of a tubular shaft that is rotatably held in the housing 102 to allow relative rotation between the drive shaft 130 and the housing 102 but is prevented from moving axially relative to the housing 102. In particular, the forward end of the drive shaft 130 has a radial flange 137 which is rotatably held within an annular recess within the housing 102.

The rearward end of the plunger 120 is engaged with the drive shaft 130 and the forward end of the plunger 120 is engaged with an internal annular flange 104 within the housing 102. In particular, the plunger 120 is received through a bore 131 in the drive shaft 130 and through the centre of the internal annular flange 104. The plunger 120 comprises a helical thread that is engaged with a corresponding helical thread within the bore 131. The plunger 120 further comprises axially extending grooves that engage with axially extending splines on the internal annular flange 104. Thus, when the drive shaft 130 rotates relative to the housing 102, the plunger 120 is constrained to move axially relative to drive shaft 130 along the helical thread in the bore 131. Alternatively, the helical thread could be provided on the internal annular flange 104 and the axially extending splines could be provided in the bore 131 to achieve the same effect. The rotational displacement of the drive shaft 130 during rotation relative to the housing 102 determines the axial displacement of the plunger 120, which determines the dose that will be delivered from the cartridge or syringe.

The forward end of the plunger 120 comprises an enlarged head 121 for pushing on a bung within the cartridge or syringe during dose delivery.

In injection device 100, the drive spring 110 is engaged between the drive shaft 130 and the housing 102 and is in the form of a torsion spring. The drive spring 110 is located in an annular recess of the drive shaft 130. One end of the drive spring 110 is rotationally fixed to the drive shaft 130 at the rearward end of the annular recess and the other end of the drive spring 110 is rotationally fixed to the housing 102 (in particular the rearward end of the internal annular flange 104). During assembly of the injection device 100, the drive spring 110 is preloaded (energised) such that prior to a first dose setting operation, the drive spring 110 has enough energy to supply the force required to perform a plurality of dose delivery operations without the user needing to input any energy into the drive spring 110. In other words, the drive spring 110 is preloaded with enough energy to advance the plunger 120 for a first dose delivery and has enough energy remaining to advance the plunger 120 for a subsequent dose delivery without the user needing to input any energy into the drive spring 110 at any stage. The drive spring 110 can be preloaded with enough energy to supply the force required to deliver substantially the entire deliverable volume of substance in the cartridge or syringe over one or more dose delivery operations without the user needing to input any energy into the drive spring 110. Thus, after the injection device 100 is assembled, and before a first dose setting operation, the drive spring 110 is applying a torque between the drive shaft 130 and the housing 102. However, when the injection device 100 is at rest, the drive shaft 130 is prevented from rotating relative to the housing 102 under the influence of the drive spring 110 by the clutch member 140.

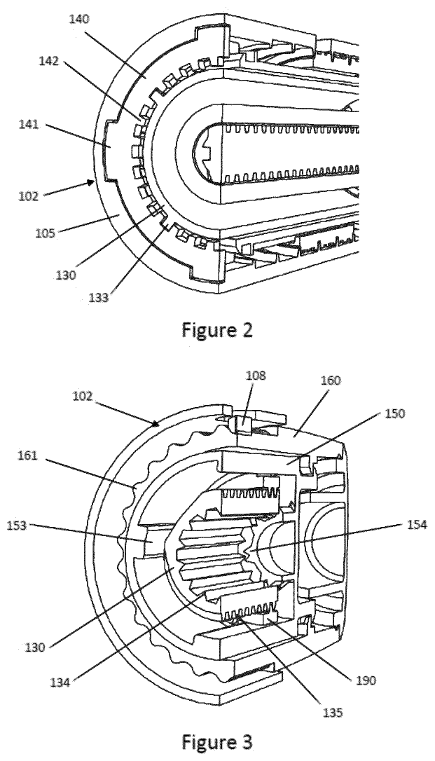

FIG. 2 shows the clutch member 140 engaged between the dose drive shaft 130 and the housing 102. The clutch member 140 is annular and located radially between the outer surface of the drive shaft 130 and the inner surface of the housing 102. The inner and outer sides of the clutch member 140, the outer surface of the drive shaft 130, and the inner surface of the housing 102 each comprise formations in the form of axially extending splines that can interlock with each other in the circumferential direction. The axially extending splines are arranged such that the clutch member 140 is in splined engagement between the drive shaft 130 and the housing 102 at a first axial position (hereinafter referred to as an engaged position) but moves out of splined engagement between the drive shaft 130 and the housing 102 when moved to a second axial position (hereinafter referred to as a disengaged position) relative to the housing 102. Thus, when the clutch member 140 is in the engaged position, the drive shaft 130 is prevented from rotating relative to the housing 102 under the influence of the drive spring 110 (thereby holding the drive spring 110 in an energised state) and when the clutch member 140 is in the disengaged position, the drive shaft 130 is free to rotate relative to the housing 102 under the influence of the drive spring 110 (i.e. the drive spring 110 is released).

The dose setting member 150 is generally in the form of a tubular sleeve located radially between the drive shaft 130 and the housing 102. The dose setting member 150 extends axially beyond the rearward end of the drive shaft 130. The dose setting member 150 is coupled to the clutch member 140 by a snap fit connection 151 that allows the dose setting member 150 and the clutch member 140 to rotate relative to each other but allows the dose setting member 150 to axially move the clutch member 140 between its engaged and disengaged positions.

FIG. 3 shows a cross-section view of the injection device 100 showing the rearward ends of the drive shaft 130 and the dose setting member 150. The drive shaft 130 and the dose setting member 150 both comprise engagement features at their rear ends. In particular, the drive shaft 130 comprises formations in the form of axially extending spines 134 and the dose setting member 150 comprises formations in the form of axially extending splines 154. The dose setting member 150 is axially moveable relative to the drive shaft 130 from a first (rearward) position in which the axially extending splines 134, 154 are not engaged to allow the dose setting member 150 to rotate relative to the drive shaft 130, to a second (forward) position in which the axially extending splines 134, 154 are engaged and interlock in the circumferential direction so that the drive shaft 130 and the dose setting member 150 are rotationally fixed relative to each other (such that they will co-rotate when one of them rotates).

The clutch member 140, drive shaft 130 and dose setting member 150 are axially arranged such that when the injection device 100 is at rest (i.e. not in a dose delivery state), the clutch member 140 is in its engaged position and the dose setting member 150 is in its first position such that the dose setting member 150 is not in engagement with the drive shaft and the clutch is preventing relative rotation between the drive shaft 130 and the housing 102. Furthermore, axial movement of the dose setting member 150 from its first position to its second position moves the clutch member to its disengaged state such that the dose setting member 150 engages with the drive shaft 130 and the drive shaft 130 is free to rotate relative to the housing under the influence of the drive spring 110.

The axial position and length of the engagement features between the dose setting member 150 and the drive shaft 130 and between the clutch member 140, the housing 102 and the drive shaft 130 can be configured such that when the dose setting member 150 is moved from its first position to its second position, the dose setting member 150 moves into engagement with the drive shaft 130 before the clutch disengages. This prevents the drive shaft 130 from rotating relative to the housing 102 during dose delivery without co-rotation with the dose setting member 150.

The dose button 160 is attached to the rearward end of the housing 102 and extends axially beyond it so that the user can operate it. The dose button 160 is rotatable and axially moveable relative to the housing 102. A return spring 170 is engaged between the dose button 160 and the housing 102 to bias the dose button 160 in a rearward direction to a first position.

The dose button 160 comprises teeth 161 spaced circumferentially about the outer surface of the dose button 160. The inner surface of the housing 102 further comprises a flexible finger 108 positioned to interact with each tooth to produce audible and/or tactile feedback when the user is rotating the dose button 160 relative to the housing 102.

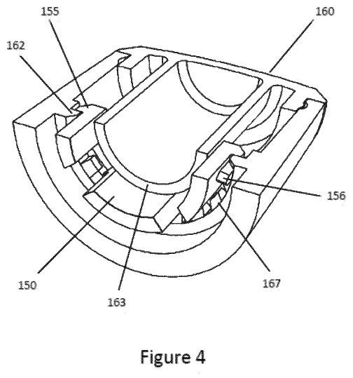

When the injection device 100 is at rest, dose button 160 is at its first (rearward) position due to the bias of the return spring 170. FIG. 4 shows a cross-section view of the dose button 160 and the rear end of the dose setting member 150 in this first position. When the dose button 160 is in its first position, the dose button 160 is engaged with the rearward end of the dose setting member 150 such that the dose button 160 is rotationally fixed relative to the dose setting member 150 but can move axially relative to the dose setting member 150. In particular, the rearward end of the dose setting member 150 comprises formations in the form of axially extending splines 156 that interlock in the circumferential direction with formations in the form of axially extending splines 167 on the dose button 160. Thus, when the dose button 160 is at its first position, rotation of the dose button 160 relative to the housing 102 will rotate the dose setting member 150 together with the dose button 160.

The dose button 160 can be pushed forwardly relative to the dose setting member 150 to axially move the dose button 160 from its first position to a second position in which the axially extending splines on the dose button 160 and the dose setting member 150 are disengaged so that the dose setting member 150 is able to rotate relative to the dose button 160. To axially move the dose setting member 150, the dose button 160 further comprises a forwardly projecting abutment 163 for pushing on the rearward end of the dose setting member 150. The abutment 163 is positioned such that when the dose button 160 is pushed forwardly, the axially extending splines 167 of the dose button 160 and the dose setting member 150 are disengaged before or at the same time as when the abutment 163 abuts the rearward end of the dose setting member 150. Thus, once the dose button 160 is pushed forward far enough for the abutment 163 to begin pushing the dose setting member 150 forward, the dose setting member 150 is free to rotate relative to the dose button 160.

The rearward end of the dose setting member 150 further comprises an outwardly extending radial flange 155 and the dose button 160 further comprises an inwardly extending radial flange 162. The flanges 155, 162 are engaged in the axial direction such that rearward axial movement of the dose button 160 (e.g. under the influence of the return spring 170) will pull the dose setting member 150 with it in the rearward direction.

When the injection device 100 is at rest and the dose button 160 is pushed forward, the dose setting member 150 is axially moved from its first position to its second position and hence the clutch member 140 is axially moved from its engaged position to its disengaged position. Thus, the dose button 160 can perform the function of setting a dose (by rotating the dose button 160) and triggering dose delivery (by pushing the dose button 160) via its interactions with the dose setting member 150.

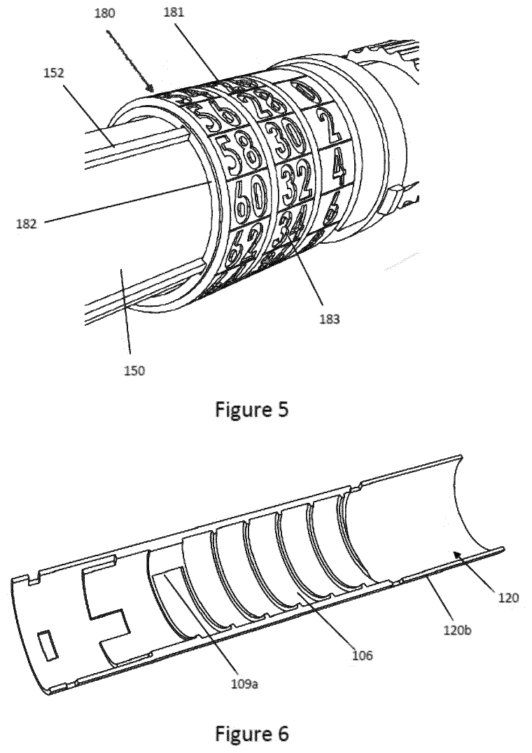

FIG. 5 shows the control member 180 in engagement with the dose setting member 150 (the housing 102 is not shown). The control member 180 is engaged radially between the dose setting member 150 and the housing 102. The purpose of the control member 180 is to limit the rotational displacement of the drive shaft 130 during dose delivery according to the dose set by the user. The control member 180 is engaged between the dose setting member 150 and the housing 102 such that it is rotationally fixed and axially movable relative to the dose setting member 150 and is constrained to move along a helical path relative to the housing 102. In particular, the inner surface of the control member 180 comprises formations in the form of axially extending splines 182 that interlock in the circumferential direction with formations in the form of axially extending splines 152 on the outer surface of the dose setting member 150. Furthermore, the outer surface of the control member 180 comprises a helical thread 181 that engages with a corresponding helical thread 106 on the inner surface of the housing 102. Thus, when the dose setting member 150 is rotated relative to the housing 102, the control member 180 moves along the helical thread 106 of the housing 102 in the axial direction.

FIG. 6 is a cross-section view showing the inner surface of the housing 102. The helical thread 106 of the housing 102 has a forward terminating end. The forward terminating end provides a zero dose stop surface 109a that prevents the control member 180 from moving forward along the helical thread 106 past the zero dose stop surface 109a. A maximum dose stop surface 109b is provided by an internal component positioned at the rearward end of the helical thread 106 that prevents the control member 180 from moving rearwardly along the helical thread 106 past the maximum dose stop surface 109b.

Alternatively, the zero dose stop surface 109a could be provided by a surface of an internal component or the housing, and the maximum dose stop surface 109b could be provided by a rearward terminating end of the helical thread 106.

The handedness of the helical thread 106 and the direction of the torque applied by the drive spring 110 between the housing 102 and the drive shaft 130 are chosen such that rotation of the drive shaft 130 relative to the housing under the influence of the drive spring 110 when the clutch member 140 is disengaged rotates the dose setting member 150 causes the control member 180 to move toward the zero dose stop surface 109a until the control member abuts the zero dose stop surface 109a. Thus, the zero dose stop surface 109a corresponds to a zero dose (i.e. when no dose has been set, the control member 180 abuts the zero dose stop surface 109a). The dose setting member 150 therefore sets a dose by setting the position of the control member 180 relative to the stop surface 109a, which determines the maximum rotational displacement of the drive shaft 130 when the clutch is disengaged, which determines the maximum axial displacement of the plunger 120.

The maximum dose stop surface 109b provides a maximum limit to the position of the control member 180 relative to the zero dose stop surface 109a. Thus, the maximum dose stop surface 109b provides a limit on the maximum dose that can be set for a single dose delivery. The control member 180 also provides an indication of the dose set by the user by way of a sequence of numbers 183 marked and arranged helically on the outer surface of the control member 180. The housing 102 further comprises a window 107 (which may be in the form of an aperture or a transparent portion) through which a number indicating the set dose can be seen.

The numbers 183 on the control member 180 are arranged such that when the control member 180 abuts against the zero dose stop surface 109a, the control member 180 indicates a dose of zero through the window in the housing 102.

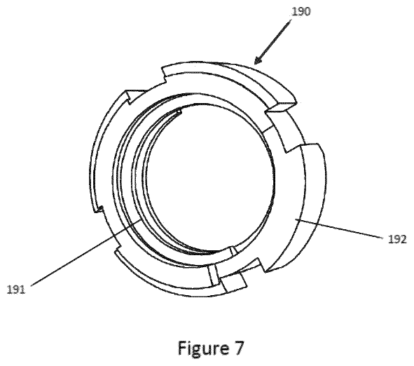

FIG. 7 shows the stop member 190. The stop member 190 is in the form of an annular nut and is engaged radially between the drive shaft 130 and the dose setting member 150. The outer surface of the drive shaft 130 comprises a helical thread 135 (shown in FIGS. 1 and 3), which engages with a corresponding helical thread 191 on the inner surface of the stop member 190. The inner surface of the dose setting member 150 comprises formations in the form of axially extending splines 153 (shown in FIG. 3) that interlock in the circumferential direction with formations in the form of axially extending splines 192 on the outer surface of the stop member 190 in order to prevent relative rotation between the stop member 190 and the dose setting member 150 but allow relative axial movement. Thus, when the dose setting member 150 is rotated relative to the drive shaft 130, the stop member 190 is constrained to travel along the helical thread 135 in the axial direction but if the drive shaft 130 and dose setting member 150 rotate together (with no relative rotation between them), there is no force to drive the stop member 190 to travel along the helical thread 135 and therefore the stop member 190 will not travel along the helical thread 135.

The helical thread 135 terminates at a forward end. This forward terminating end provides a last dose stop surface 136 that prevents the stop member 190 from moving forward along the helical thread past the last dose stop surface 136. Thus, the position of the last dose stop surface 136 determines the maximum distance along the helical thread 135 that the stop member 190 can travel toward the last dose stop surface 136. The position of the last dose stop surface 136 relative to the initial position of the stop member 190 during assembly of the device 100 and prior to a first dose setting operation by the user is predetermined and can be chosen to correspond to the total volume of substance deliverable from the cartridge or syringe.

The handedness of the helical thread 106 on the housing 102 and the handedness of the helical thread 135 of the drive shaft 130 are chosen such that rotation of the dose setting member 150 relative to the housing 102 in one direction causes the control member 180 to move away from the zero dose stop surface 109a and the stop member 190 to move toward the last dose stop surface 136; and rotation of the dose setting member 150 relative to the housing 102 in the opposite direction causes the control member 180 to move toward the zero dose stop surface 109a and the stop member 190 to move away from the last dose stop surface 136.

Alternatively, the outer surface of the stop member 190 could be threadedly engaged with the inner surface of the dose setting member 150 and the inner surface of the stop member 190 could be in splined engagement the outer surface of the drive shaft 130, with a stop surface for preventing linear movement along the drive shaft 130 after the stop member 190 has travelled a predetermined linear distance.

The interactions between the components of the injection device 100 will now be described in more detail with respect to use of the device.

The injection device 100 is operable between two main states—a dose setting state for setting a dose and a dose delivery state for delivering the set dose.

When the injection device 100 is at rest, the injection device 100 is in the dose setting state for setting a dose. In the dose setting state, the clutch member 140 is in its engaged position, the dose setting member 150 is in its first position and the dose button 160 is in its first position. The preloaded drive spring 110 is applying a torque between the drive shaft 130 and the housing 102, but the drive shaft 130 is prevented from rotating relative to the housing 102 (which would cause the plunger 120 to advance) because the clutch member 140 is engaged between the drive shaft 130 and the housing 102. Thus, the torque from the drive spring 110 is being transmitted between two parts of the housing that cannot move relative to each other and therefore there is no resultant movement. Furthermore, the control member 180 is at the zero dose stop surface 109a and the stop member 190 is at an initial position away from the last dose stop surface 136.

To set a dose, the dose button 160 is rotated in a particular direction relative to the housing 102. The dose setting member 150 rotates together with the dose button 160 because the dose button 160 is rotationally fixed relative to the dose setting member 150 when the dose button 160 is in the first position. Rotation of the dose setting member 150 causes the control member 180 to advance rearwardly along the helical thread of the housing 102 away from the zero dose stop surface 109a. The numbers 183 on the control member 180 can indicate to the user the dose (e.g. the number of units) that has been dialled so far because the control member 180 moves relative to the window in the housing 102 when the dose setting member 150 is rotated. The audible and/or tactile feedback provided by the finger 108 also helps the user to identify each dose increment as the dose button 160 is rotated. At the same time, the stop member 190 advances forwardly along the helical thread of the drive shaft 130 toward the last dose stop surface 136. If too high a dose has been set, the dose button 160 can be rotated in the opposite direction, which will move the control member 180 back toward the zero dose stop surface 109a and move the stop member 190 away from the last dose stop surface 136.

Furthermore, when the dose setting member 150 and dose button 160 are in their first positions, there is no torque transmission path from the drive spring 110 to the dose setting member 150 or dose button 160 because the dose setting member is not engaged with the drive shaft 130. Thus, the dose button 160 and dose setting member 150 can rotate relative to the housing 102 to set a dose (via movement of the control member 180) without doing any work against the drive spring 110. In other words, in the dose setting state and during a dose setting operation, the force of the drive spring 110 is completely isolated from the dose setting member 150 and the dose button 160.

Once the dose has been set, the dose button 160 is moved axially forward relative to the housing 102 to its second position and is held there by the user. The injection device 100 is now in its dose delivery state. Movement of the dose button 160 to the second position causes the dose button 160 to rotationally disengage from the dose setting member 150 and moves the dose setting member 150 axially forward to its second position. Movement of the dose setting member 150 to its second position causes the dose setting member 150 to move into splined engagement with the drive shaft 130, and causes the clutch member 140 to move to its disengaged position. Thus, in the dose delivery state, the dose setting member 150 can rotate relative to the dose button 160, the drive shaft 130 can rotate relative to the housing 102 and the dose setting member 150 is rotationally fixed relative to the drive shaft 130.

Once the clutch member 140 has moved to its disengaged position, the drive shaft 130 is now permitted to rotate relative to the housing 102 under the influence of the drive spring 110. This causes the plunger 120 to advance forwardly relative to the housing 102, and move a bung inside the cartridge or syringe to start dispensing a dose. Furthermore, because the drive shaft 130 is now in splined engagement with the dose setting member 150, the drive shaft 130 and the dose setting member 150 rotate together relative to the housing 102. This causes the control member 180 to advance forwardly along the helical thread 106 of the housing 102 toward the zero dose stop surface 109a. However, because the drive shaft 130 and dose setting member 150 are rotating together during the dose delivery operation, the stop member 190 does not advance along the helical thread 135 of the drive shaft 130 and therefore remains at the same position relative to the last dose stop surface 136.

Once the control member 180 reaches the zero dose stop surface 109a, the control member 180 is prevented from moving further forward relative to the housing 102 and therefore the dose setting member 150 is prevented from rotating any further relative to the housing. This also stops the drive shaft 130 from rotating relative to the housing 102 because the drive shaft 130 and the dose setting member 150 are rotationally fixed relative to each other. As a result, the plunger 120 stops advancing relative to the housing 102 and the set dose is considered to be delivered.

Once the dose has been delivered, the dose button 160 can be released by the user. The return spring 170 moves the dose button 160 rearwardly from its second position to its first position. This causes the dose setting member 150 to move rearwardly from its second position to its first position and the clutch member 140 to move rearwardly from its disengaged position to its engaged position. Thus, the drive shaft 130 is prevented from rotating relative to the housing 102 under the influence of the drive spring 110 (which is still energised due to the preload). In this post-delivery state, the control member 180 has returned to its original position abutting the zero dose stop surface 109a. However, the stop member 190 is still at the same position relative to the last dose stop surface 136 from just after the dose was set because the stop member 190 could not advance along helical thread 135 during dose delivery.

It can therefore be seen that the position of the stop member 190 relative to the last dose stop surface 136 at any point in time corresponds to the cumulative doses that have been set and delivered up to that point. Subsequent dose settings and dose deliveries will act to move the stop member 190 closer and closer to the last dose stop surface 136 until the stop member 190 abuts the last dose stop surface 136. Once the stop member 190 abuts the last dose stop surface 136, the stop member 190 cannot move any further along the helical thread 135, which prevents further rotation of the dose setting member 150 (by the dose button 160) relative to the drive shaft 130 in the dose setting direction. This also prevents relative rotation between the dose setting member 150 and the housing 102 because the drive shaft 130 is rotationally fixed relative to the housing 102 in the dose setting state due to the clutch member 140. Therefore, once the stop member 190 abuts the last dose stop surface 136, the dose button 160 can no longer be rotated relative to the housing 102 to increase the set dose. Given that the distance along the helical thread 135 between the last dose stop surface 136 and the initial position of the stop member 190 (from before the first dose setting operation) corresponds to the total volume of substance that can be delivered from the cartridge or syringe, the stop member 190 acts to prevent the user from setting a dose that exceeds the total volume that is remaining in the cartridge or syringe.

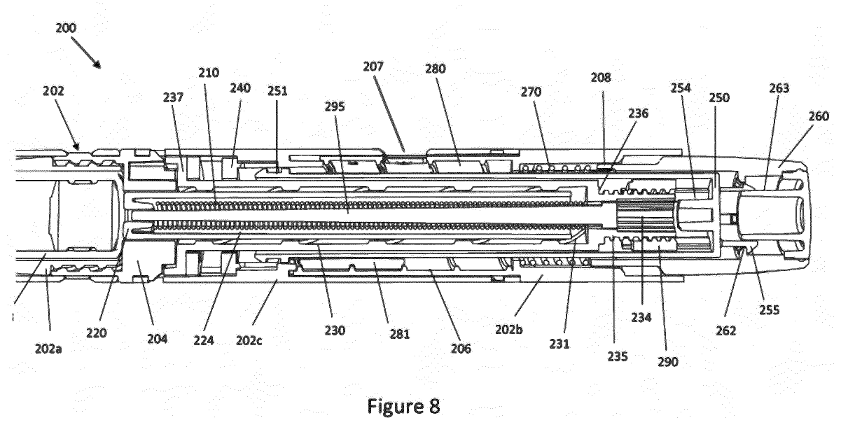

FIG. 8 shows a cross-section view of another injection device 200. The parts of injection device 200 that provide the same or a similar function to parts of injection device 100 are labelled with the same reference numerals as those parts, except that the reference numerals are prefixed with the number “2”, rather than the number “1”.

The drive mechanism of injection device 200 comprises most of the same components as the drive mechanism of injection device 100 and which interact in the same way, and therefore these similarities will not be described in detail again. However, there are some differences, which are explained below.

In injection device 200, the drive spring 210 is in the form of a compression spring 210 rather than a torsion spring. The compression spring 210 is co-axial with the longitudinal axis of the housing 202 and is arranged axially between the drive shaft 230 and the plunger 220. In particular, the compression spring 210 is engaged between a forward-facing surface of the drive shaft 230 and a rearward-facing surface of the plunger 220. The forward-facing surface of the drive shaft 230 is located within the bore 231 of the drive shaft 230 and the rearward-facing surface of the plunger 220 is located within a rearwardly extending bore 224 in the plunger 220 in which the compression spring 210 is received.

The compression spring 210 exerts a linear force in the axial direction between the drive shaft 230 and the plunger 220. This acts to push the plunger 220 forwardly out of the bore of the drive shaft 230, but due to the threaded engagement between the drive shaft 230 and the plunger 220, the plunger 220 can only move forwardly if the drive shaft 230 is able to rotate relative to the housing. Thus, when the clutch member 240 is in the engaged position, the plunger 220 is prevented from advancing forwardly relative to the housing 202 under the influence of the compression spring 210; but when the clutch member 240 is in the disengaged position, the force applied between the plunger 220 and the drive shaft 230 by the compression spring 210 is resolved by the drive shaft 230 rotating relative to the housing 202 and the plunger 220 advancing forwardly relative to the housing 202. The drive shaft 230 is therefore indirectly rotated relative to the housing 202 by the drive spring 210 in injection device 200.

Similar to the torsion spring of injection device 100, the compression spring 210 is preloaded during assembly of the injection device 200 such that prior to a first dose setting operation, the drive spring has enough energy to supply the force required to perform a plurality of dose delivery operations without the user needing to input any energy into the compression spring 210. The compression spring 210 can be preloaded with enough energy to supply the force required to deliver substantially the entire deliverable volume of substance in the cartridge or syringe over one or more dose delivery operations without the user needing to input any energy into the compression spring 210.

The compression spring 210 can optionally be preloaded with torsion in addition to compression in order to assist in overcoming any friction during relative rotation between the plunger 220, drive shaft 230, control member 280 and the housing 202 that would otherwise interrupt smooth operation during dose delivery. Given that the torque provided by the torsion preload is intended to supplement the linear force provided by the compression spring 210, the torsion preload may provide a torque less than the torque required to deliver the total deliverable volume of substance in the cartridge or syringe.

Features of the device 200 and a method relating to torsioning (twisting) and compression of the compression spring 210 will now be described.

FIG. 9 shows the compression spring 210 used in the injection device 200. The rearward end of the compression spring 210 is provided with an integral axially extending end formation 210a and the forward end of the compression spring 210 is provided with an integral axially extending end formation 210b. The compression spring 210 is longer than the bore 231 of the drive shaft 230 when the compression spring 210 is in a relaxed (i.e. uncompressed) state.

As shown in FIG. 8, the compression spring 210 is arranged on a pin 295 extending axially through the centre of the coils of the spring 210. The pin 295 is optional, but is provided to help stabilise the compression spring 210 and to prevent the compression spring 210 from bending off-axis.

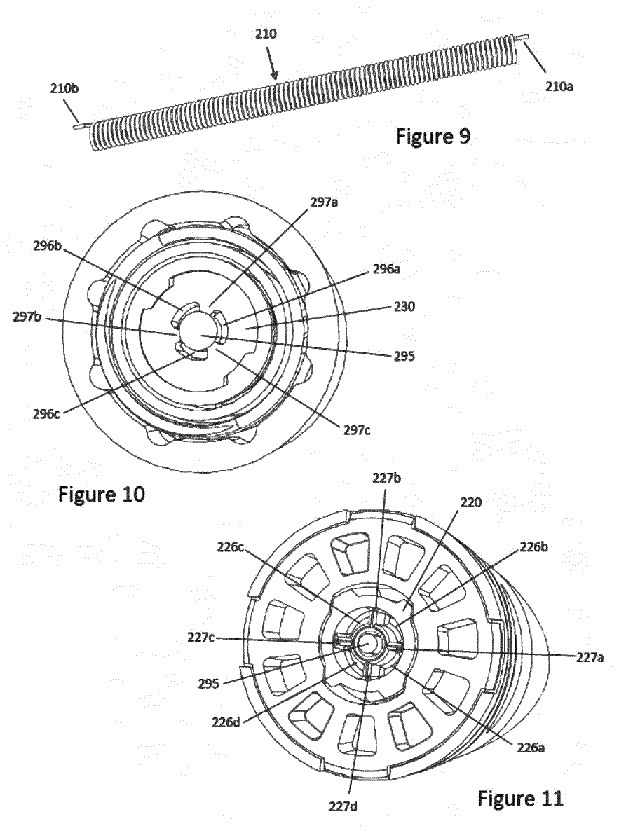

FIG. 10 shows the forward-facing surface of the drive shaft 230 that is engaged with the rearward end of the compression spring 210. The forward-facing surface of the drive shaft 230 is provided with three seats 296a-296c arranged circumferentially about the longitudinal axis. Each seat 296a-296c is in the form of a recess for receiving the rearward end formation 210a of the compression spring 210. The recesses are separated from each other in the circumferential direction by circumferential walls 297a-297c. From FIG. 10, it can be seen that the outer surface of the pin 295 forms the inner radial wall of each recess. The inner radial wall of each recess could instead be formed by an integral surface of the drive shaft 230; however, by allowing the pin 295 to form the inner radial wall, the pin 295 can be made larger in diameter, which allows the pin 295 to stabilise the compression spring 210 more effectively. It can also be seen that a forward-facing surface at the rearward end of the pin 295 forms the base of each recess; however, the base of each recess could also be formed by an integral surface of the drive shaft 230.

FIG. 11 shows the rearward-facing surface of the plunger 220 that is engaged with the forward end of the compression spring 210. The rearward-facing surface of the plunger 220 is provided with four seats 226a-226d arranged circumferentially about the longitudinal axis. Each seat is in the form of a recess for receiving the forward end formation of the compression spring 210. The recesses are separated in the circumferential direction by circumferential walls 227a-227d.

During assembly of the injection device 200, the pin 295 is inserted into the bore 231 of the drive shaft 230 from the rearward side of the device. The compression spring 210 is then inserted into the bore 231 of the drive shaft 230 and over the pin 295 from the forward side of the device until the rearward end formation 210a of the compression spring 210 is received in one of the seats 296a-296c. The particular seat in which the rearward end formation 210a of the spring 210 is received does not matter, and will depend on the angular orientation of the spring 210 relative to the drive shaft 230 when the spring is inserted. The circumferential walls 297a-297c between each seat 296a-296c may be shaped or bevelled such that if the rearward end formation 210a of the spring 210 is not immediately received in one of the seats 296a-296c, it will be diverted into one of the seats 296a-296c as the spring 210 is inserted further rearward into the drive shaft 230. Thus, the rearward end formation 210a of the spring 210 can be received in a seat 296a-296c of the drive shaft, regardless of the angular orientation of the spring 210 relative to the drive shaft 230 during assembly.

The plunger 220 is then moved in the rearward direction toward the drive shaft 230 such that the forward end of the compression spring 210 is received into the bore 224 of the plunger 220. Rearward movement of the plunger 220 continues until the forward end formation 210b of the compression spring 210 is received in one of the seats 226a-226d of the plunger 220. The particular seat in which the forward end formation 210b of the spring 210 is received does not matter, and will depend on the angular orientation of the spring 210 relative to the plunger as the spring is received in the bore 224 of the plunger 220. The circumferential walls 227a-227d may be shaped or bevelled such that if the forward end formation 210b of the spring 210 is not immediately received in one of the seats 226a-226d, it will be diverted into one of the seats 226a-226d as the spring 210 is inserted further into the bore 224 of the plunger 220. Thus, the forward end formation 210b of the spring 210 can be received in a seat 226a-226d of the plunger 220, regardless of the angular orientation of the spring 210 relative to the plunger 220 during assembly.

The plunger 220 is then moved further rearward relative to the drive shaft 230 until the helical thread on the outer surface of the plunger 220 and the helical thread on the inner surface of the drive shaft 230 begin to engage. At this point, the plunger 220 is rotated relative to the drive shaft 230 so that the plunger 220 becomes threadedly engaged with the drive shaft 230 and therefore further relative rotation between the plunger 220 and the drive shaft 230 causes the plunger 220 to move axially rearward relative to the drive shaft 230.

As the plunger 220 moves axially rearward relative to the drive shaft 230, the compression spring 210 becomes more and more compressed between the plunger 220 and the drive shaft 230 and the end formations 210a, 210b of the spring 210 engage with (push against) their respective seats in the axial direction. Furthermore, the end formations 210a, 210b of the spring 210 may also rotate in their respective seats 296a-296c, 226a-226d until they engage with (abut against) the side of a respective circumferential wall 227a-227d, 297a-297c. This engagement restricts or resists relative rotation between the forward end formation 210b and the drive shaft 230 and between the rearward end formation 210a and the plunger 220 as the plunger 220 rotates relative to the drive shaft 230. Thus, relative rotation between the plunger 220 and the drive shaft 230 causes the forward and rearward ends of the spring 210 to rotate relative to each other, which torsions (twists) the compression spring 210. Thus, the action of threadedly engaging the plunger 220 into the drive shaft 230 by relative rotation causes the compression spring 210 to be simultaneously compressed and torsioned between the plunger 220 and the drive shaft 230.

The exact number of seats of the plunger 220 and the drive shaft 230 is not essential. Fewer seats means larger recesses for the end formations of the spring 210 to fall into. However, this also means that a larger rotational displacement between the plunger 220 and the spring 210 may be required during assembly to torsion the spring 210. The above-described method of compressing and torsioning a compression spring between a drive shaft and a plunger threadedly engaged with the drive shaft is not limited to the specific injection device 200 and is generally applicable to any injection device that uses a rotary drive mechanism comprising a drive shaft threadedly engaged with a plunger and a compression spring for driving the plunger.

In its assembled state, the injection device 200 therefore comprises a plunger 220 threadedly engaged with a drive shaft 230, and a compression spring 210 axially engaged between the plunger 220 and the drive shaft 230. The compression spring 210 is in a compressed and torsioned state and acts to apply a linear force and a torque between the plunger 220 and the drive shaft 230. A forward end of the compression spring 210 is engaged in a seat 226a-226d on the plunger 220, and a rearward end of the compression spring 210 is engaged in a seat 297a-297c on the drive shaft 230. As with the above-described method, this drive mechanism is not limited to being used in the specific injection device 200 and is generally applicable to any injection device that uses a rotary drive mechanism including a drive shaft threadedly engaged with a plunger and a compression spring for driving the plunger.

Another difference between injection devices 100 and 200 is that the housing is split into three portions—a forward portion 202a, a rearward portion 202b and an intermediate portion 202c arranged between the forward portion 202a and the rearward portion 202b. The helical thread 206 of the housing 202 is located on the rearward portion 202b and a rearward-facing surface of the intermediate portion 202c provides the maximum dose stop surface 209b for the control member 208. However, this housing configuration is not essential and the injection device 200 could have the same housing configuration as injection device 100 and vice versa.

Injection device 200 is particularly suitable for the delivery of large doses. If a torsion spring were to be used to deliver a large dose, the pitch of the helical threads of the drive shaft 230 and plunger 220 would be too large to fit in a device of reasonable length. Thus, the use of a compression spring 210 allows large doses to be delivered using a shorter device compared to using a torsion spring.

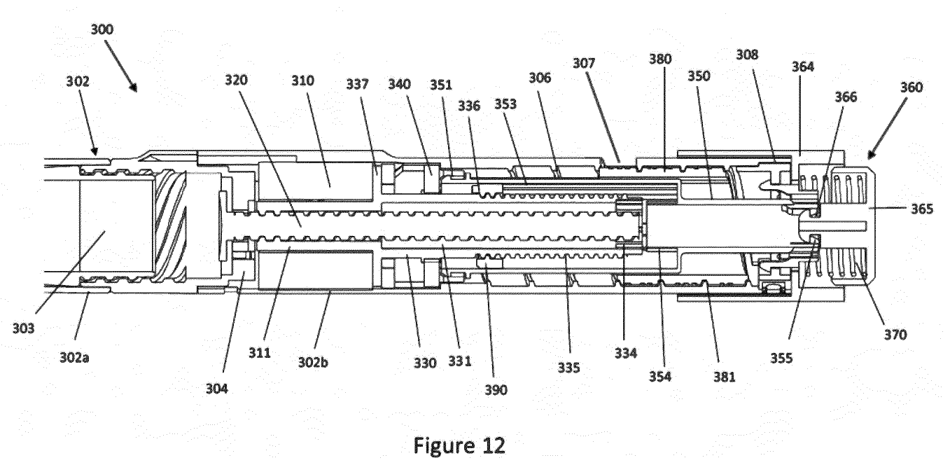

FIG. 12 shows a cross-section view of another injection device 300. The parts of injection device 300 that provide the same or a similar function to parts of injection device 100 are labelled with the same reference numerals as those parts, except that the reference numerals are prefixed with the number “3”, rather than the number “1”.

The drive mechanism of injection device 300 comprises most of the same components as the drive mechanism of injection device 100 and which interact in the same way, and therefore these similarities will not be described in detail again. However, there are some differences, which are explained below.

In injection device 300, the drive spring 310 is in the form of a power spring 310, rather than a torsion spring. The power spring 310 comprises a band of metal that is wound around a central arbor (shaft) 311 that is co-axial with the longitudinal axis of the housing 302. The power spring 310 is arranged radially between the drive shaft 330 and the housing 302. The outer end of the power spring 310 is attached to the housing 302 and the inner end of the power spring 310 is attached to the outer surface of the arbor 311 such that a torque is applied by the power spring 310 between the housing 302 and the arbor 311. The arbor 311 is formed integrally with or is attached to the forward end of the drive shaft 330, such that the arbor 311 is rotationally fixed relative to the drive shaft 330. Thus, the torque applied by the power spring 310 acts to rotate the drive shaft 330 relative to the housing 302. The bore 331 of the drive shaft 330 extends axially through the arbor 311 such that the plunger 320 is received within the drive shaft 330 and the arbor 311.

Similar to the torsion spring of injection device 100, the power spring 310 is preloaded during assembly of the injection device 300 such that prior to a first dose setting operation, the drive spring 310 has enough energy to supply the force required to perform a plurality of dose delivery operations without the user needing to input any energy into the power spring 310. The power spring 310 can be preloaded with enough energy to supply the force required to deliver substantially the entire deliverable volume of substance in the cartridge or syringe over one or more dose delivery operations without the user needing to input any energy into the drive spring 310.

Another difference between injection devices 100 and 300 is the dose button 360. In injection device 100, the dose button 160 provides two functions—rotating the dose button 160 sets a dose and axially pushing the dose button 160 delivers the set dose. In injection device 300, these two functions have been split into two different components—a dial 364 and a firing button 365.

The dial 364 circumferentially surrounds the rearward end of the housing 302 and is rotatable relative to the housing 302. The dial 364 is in splined engagement with the rearward end of the dose setting member 350 when the dose setting member 350 is in its first position so that the dial 364 is rotationally fixed relative to the dose setting member 350. Rotation of the dial 364 performs the same function as rotation of the dose button 160 of the device 100 when the dose button 160 is in its first position.

The firing button 365 is located at the rearward end of the device and is axially moveable relative to the dial 364 and housing 302. The firing button 365 is biased rearward by the return spring 370, which is engaged between a rearward facing surface of the dial 364 and a forward facing surface of the firing button 365. The firing button 365 is axially engaged with the rear end of the dose setting member 350 such that the firing button 365 is axially fixed relative to the dose setting member 350.

In particular, the firing button 365 comprises an annular recess 366 that receives an inwardly extending radial flange 355 of the dose setting member 350. Thus, the radial flange 355 of the dose setting member 350 is bound on both sides in the axial direction by surfaces of the firing button 365. Thus, forward axial movement of the firing button 365 will move the dose setting member 350 forward and rearward axial movement of the firing button 365 will move the dose setting member 350 rearward. Forward movement of the firing button 365 performs the same function as forward movement of the dose button 160 of the device 100, including pushing the dose setting member 350 out of splined engagement with the dial 364.

The inner surface of the dial 364 comprises teeth 361 that interact with a finger 308 on the outer surface of the housing to produce audible and/or tactile feedback when the dial 364 is rotated relative to the housing.

Such a dose button arrangement is not limited to injection device 300 and could also be used in injection devices 100 and 200. Similarly, the dose buttons of injection devices 100 and 200 could be used in injection device 300.

A power spring 310 differs from a torsion spring or a compression spring in that the torque output is mostly independent from the angular displacement between the ends of the power spring 310. This allows for a dose delivery rate that is approximately constant regardless of how much energy is remaining in the power spring 310, which some users may find preferable. An injection device using a power spring 310 can also be made shorter than injection devices that use a torsion spring or a compression spring, at the potential expense of being wider.

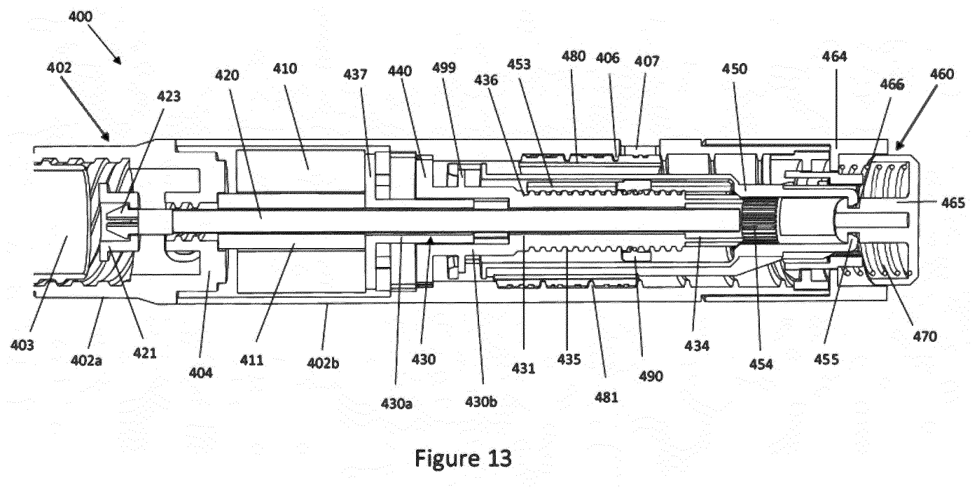

FIG. 13 shows a cross-section view of an injection device 400. Injection device is 400 is a variant of injection device 300 and also uses a power spring 410 as the drive spring, which is also preloaded as described above. The parts of injection device 400 that provide a similar function to parts of injection device 300 are labelled with the same reference numerals as those parts, except that the reference numerals are prefixed with the number “4”, rather than the number “3”.

The drive mechanism of injection device 400 comprises most of the same components as the drive mechanism of injection device 300 and which interact in the same way, and therefore these similarities will not be described in detail again. However, there are some differences, which are explained below.

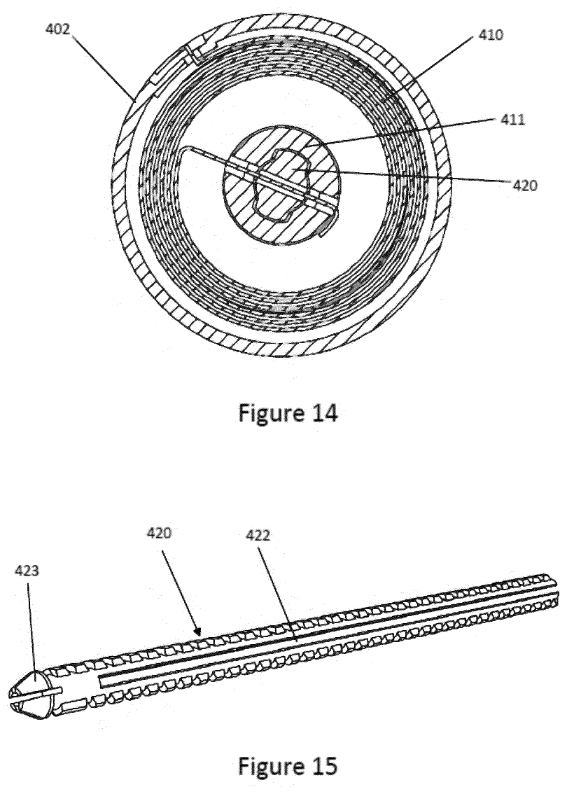

In injection device 400, an outer end of the power spring 410 is attached to the housing and the inner end of the power spring 410 is attached to the arbor 411. FIG. 14 shows a cross-section view of the injection device 400 through the diameter of the power spring 410. In contrast to the power spring 310 of injection device 300, the inner end of the power spring 410 extends through the diameter of the arbor 411, rather than extending around the outer surface of the arbor 411. This configuration can be cheaper and easier to manufacture than the configuration of power spring 310. Furthermore, the configuration of power spring 410 can allow for a stronger attachment to be made between the inner end of the power spring 410 and the arbor 411 compared to the configuration of power spring 310. In other words, if the inner end of the power spring 410 is attached to the outer surface of the arbor 411 (as it is in power spring 310), a larger diameter arbor may need to be used if the attachment is not strong enough to withstand the desired preload. By extending the inner end of the power spring 410 through the diameter of the arbor 411, a stronger attachment can be made with a smaller diameter arbor and therefore the injection device 400 can be made narrower for a given preload.

FIG. 15 shows a perspective view of a plunger 420 used in injection device 400. In order to allow the plunger 420 to be received through the bore of the arbor 411, the plunger 420 comprises a slot 422 through the diameter of the plunger 420 that axially extends from the rearward end of the plunger 420 toward the forward end of the plunger 420. The slot in the plunger 420 receives the inner end of the power spring 410 such that the plunger 420 and the inner end of the spring can both occupy the space within the bore of the arbor 411. The plunger 420 is also in keyed engagement with the arbor 411 to prevent the plunger 420 from rotating relative to the arbor 411 (shown in FIG. 14).

Given that the inner end of the spring extends through the diameter of the arbor 411 and the plunger 420, and that the plunger 420 is in keyed engagement with the arbor 411, then the plunger 420 will rotate together with the arbor 411/drive shaft 430. This is in contrast to injection devices 100, 200 and 300 in which the plunger is in threaded engagement with the drive shaft and can rotate relative to the drive shaft. Therefore, in order to advance the plunger 420 when the arbor 411/drive shaft 430 rotates relative to the housing, the forward end of the plunger 420 is threadedly engaged with the internal annular flange 440 of the housing 402, rather than being in keyed engagement with the internal annular flange 440.

Due to this arrangement, forward advancement of the plunger 420 is accompanied by rotation of the plunger 420. In order to avoid rotating the cartridge or syringe bung during dose delivery, the plunger 420 is provided with an arrowhead 423 at its forward end and a rotatable end cap 421 engaged between the rear surface of the arrowhead 423 and one of the protruding formations (visible in FIG. 15) on the plunger 420 that provide the threaded engagement with the internal annular flange 440. With this arrangement, when the shaft of the plunger 420 rotates during forward advancement of the plunger 420, the end cap 421 will not rotate but will move axially forward with the plunger 420. Thus, when the plunger 420 advances forward during dose delivery, the end cap 421 will push the cartridge or syringe bung forwardly without rotation.

Another difference between injection device 400 and injection devices 100, 200 and 300 is the arrangement of the drive shaft 430 and clutch member 440.

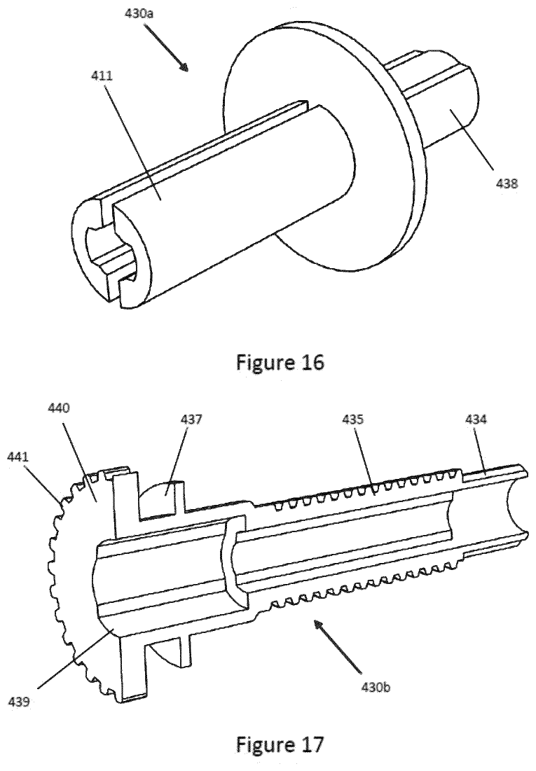

In injection device 400, the drive shaft 430 is split into two separate components—a forward drive shaft portion 430a and a rearward drive shaft portion 430b. FIG. 16 shows a perspective view of the forward drive shaft portion 430a and FIG. 17 shows a cross-section view of the rearward drive shaft portion 430b. The forward drive shaft portion 430a is formed integrally with or is attached to the arbor 411 of the power spring 410 so that it is rotationally fixed relative to the arbor 411. The rearward drive shaft portion 430b is formed integrally with or is attached to the clutch member 440 so that the rearward drive shaft portion 430b is axially fixed relative to the clutch member 440. The rearward drive shaft portion 430b comprises a helical thread in engagement with the stop member 490 and formations in the form of axially extending splines 434 for engaging with the dose setting member 450 when the dose setting member 450 is moved to its second position.

The rearward drive shaft portion 430b is axially moveable relative to the forward drive shaft portion 430a and is in keyed engagement with the forward drive shaft portion 430b to prevent relative rotation between the two drive shaft portions. In particular, diametrically opposed protrusions 438 on the forward drive shaft portion 430a engage with grooves 439 on the rearward drive shaft portion 430b. Hence, the torque applied by the power spring 410 between the housing 402 and the forward drive shaft portion 430a is transmitted to the rearward drive shaft portion 430b. However, the clutch member 440 of the rearward drive shaft portion 430b is engaged with the housing 402 when the clutch member 440 is in the engaged position to prevent relative rotation between the rearward drive shaft portion 430b and the housing 402. Thus, because the forward drive shaft portion 430a is rotationally fixed relative to the rearward drive shaft portion 430b, the forward drive shaft portion 430a is prevented from rotating relative to the housing 402 under the influence of the power spring 410 when the clutch member 440 is engaged.

The dose setting member 450 is axially engaged with an outer flange 499 on the rearward drive shaft portion 430b. Movement of the dose setting member 450 from its first position to its second position (by pushing the firing button 465 forward) moves the rearward drive shaft portion 430b and clutch member 440 from the engaged position to the disengaged position. During this movement, the rearward drive shaft portion 430b remains in keyed engagement with the forward drive shaft portion 430a. Thus, when the clutch member 440 moves to its disengaged position, the forward drive shaft portion 430a and rearward drive shaft portion 430b are free to rotate together relative to the housing 402 under the influence of the power spring 410, together with the dose setting member 450 which has moved into splined engagement with the rearward drive shaft portion 430b.

By splitting the drive shaft 430 in this way, the injection device 400 may be easier to assemble than if the drive shaft were not split. This is because when assembling the device, the angular positions of the components, in particular the relative angular positions between the stop member 490, drive shaft 430, and dose setting member 450 must be maintained as the components are being inserted into the housing. If the drive shaft were formed as a single component with the power spring 410 attached at the forward end, the power spring 410 (which is trying to expand radially outward) makes it difficult for the correct angular positions to be maintained as the components are being inserted into the housing 402. Thus, by splitting the drive shaft 430 into forward and rearward portions 430a, 430b, the power spring 410 and the helical thread for the stop member can be provided on different portions, so that angular alignment with the stop member can be maintained during assembly without interference from the power spring 410.

This split drive shaft arrangement is not limited to injection devices using a power spring 410 as the drive spring. This arrangement can also be used in any of the other described injection devices 100, 200 and 300 and would still provide a similar benefit to assembly. Instead of the forward drive shaft portion 430a being engaged with a power spring 410, the forward drive shaft portion 430a would be engaged with a torsion spring or a compression spring as appropriate. Furthermore, the clutch member 440 does not need to be formed integrally with or attached to the rearward drive shaft portion 430b. Instead, the clutch member 440 could be provided as a separate component (as in injection devices 100, 200, and 300) that engages between the housing 402 and either of the forward and rearward drive shaft portion 430a, 430b when in the engaged position.

1. An injection device for delivering a dose from a cartridge or syringe, the injection device comprising:
a housing for receiving the cartridge or syringe;
a drive shaft rotatably mounted within the housing;
a drive spring configured to directly or indirectly urge the drive shaft to rotate relative to the housing;
a plunger coupled to the drive shaft such that rotation of the drive shaft relative to the housing directly or indirectly causes the plunger to move axially relative to the housing for delivering a dose from the cartridge or syringe, in use;
a dose setting member moveable relative to the drive shaft between a first position for setting a dose and a second position for delivering a dose;
wherein when the dose setting member is in its first position, the drive shaft is rotationally fixed relative to the housing and the dose setting member is rotatable relative to the drive shaft to set the dose; and
wherein when the dose setting member is in its second position, the dose setting member is rotationally fixed relative to the drive shaft, and the drive shaft is permitted to rotate relative to the housing under the influence of the drive spring, such that the rotation of the drive shaft relative to the housing causes the dose setting member to rotate together with the drive shaft relative to the housing;
the injection device further comprising:
a stop member engaged between the dose setting member and the drive shaft such that relative rotation between the dose setting member and the drive shaft causes the stop member to travel relative to a first stop surface, and rotation of the dose setting member together with the drive shaft does not cause the first stop member to travel relative to the first stop surface;
wherein if the stop member abuts the first stop surface during relative rotation between the dose setting member and the drive shaft, continued travel of the stop member and hence continued relative rotation between the dose setting member and the drive shaft is prevented.

2. An injection device according to claim 1, wherein rotation of the dose setting member relative to the drive shaft in a direction to increase the set dose causes the stop member to travel toward the first stop surface.

3. An injection device according to claim 1 or claim 2, wherein the position of the stop member relative to the first stop surface corresponds to the total deliverable volume of substance remaining in the cartridge or syringe.

4. An injection device according to any one of claims 1 to 3, wherein the stop member is threadedly engaged with a first helical thread on one of the dose setting member and the drive shaft, and the stop member is rotationally fixed and axially movable relative to the other of the dose setting member and the drive shaft, such that relative rotation between the dose setting member and the drive shaft causes the stop member to move along the first helical thread.

5. An injection device according to claim 4, wherein the first stop surface is located on the first helical thread.

6. An injection device according to claim 4, wherein the first stop surface is located on the other of the dose setting member and the drive shaft.

7. An injection device according to any one of the preceding claims, further comprising a clutch member coupled to the dose setting member and moveable by the dose setting member between an engaged position in which the clutch member is engaged between the drive shaft and the housing to prevent relative rotation between the drive shaft and the housing, and a disengaged position in which the clutch member is not engaged between the drive shaft and the housing such that relative rotation between the drive shaft and the housing is permitted;
wherein the dose setting member and the clutch member are coupled such that when the dose setting member is moved between its first position and its second position, the clutch member is moved between its engaged position and its disengaged position respectively.

8. An injection device according to any one of the preceding claims, further comprising a dose button coupled to the dose setting member, wherein the dose button is rotatable relative to the housing and movable relative to the housing between a first position and a second position, and the dose button and the dose setting member are coupled such that:
when the dose button is in its first position, the dose setting member is in its first position and the dose setting member is rotationally fixed relative to the dose button;
when the dose button is in its second position, relative rotation between the dose setting member and the dose button is permitted; and
when the dose button is moved between its first position and its second position, the dose setting member is moved between its first position and second position respectively.

9. An injection device according to claim 8, further comprising a return spring configured to bias the dose button toward its first position.

10. An injection device according to any one of the preceding claims, wherein the dose setting member sets the dose by defining the maximum rotational displacement of the drive shaft during dose delivery.

11. An injection device according to claim 10, further comprising a control member engaged between the housing and the dose setting member such that relative rotation between the housing and the dose setting member causes the control member to travel relative to a second stop surface; wherein:
during dose delivery, relative rotation between the dose setting member and the housing under the influence of the drive spring causes the control member to move toward the second stop surface until the control member abuts the second stop surface, whereby continued rotation of the dose setting member relative to the housing is prevented; and
during dose setting, relative rotation between the dose setting member and the housing sets the dose by defining the position of the control member relative to the second stop surface.

12. An injection device according to claim 11, wherein the control member is threadedly engaged with a helical thread on the housing, and is rotationally fixed and axially movable relative to the dose setting member.

13. An injection device according to any one of the preceding claims, wherein the drive spring is a torsion spring.

14. An injection device according to any one of claims 1 to 12, wherein the drive spring is a compression spring.

15. An injection device according to claim 14, wherein the compression spring is axially engaged between the plunger and the drive shaft for applying a linear force between the plunger and the drive shaft.

16. An injection device according to claim 15, wherein the compression spring is configured to apply a torque between the plunger and the drive shaft in addition to the linear force.

17. An injection device according to any one of claims 1 to 12, wherein the drive spring is a power spring.

18. An injection device according to claim 17, wherein the power spring comprises an outer end attached to the housing and an inner end attached to an arbor, wherein the arbor is integrally formed with or attached to the drive shaft.

19. An injection device according to any one of the preceding claims, wherein when the dose setting member is in its first position, the dose setting member is completely isolated from the force of the drive spring.

20. An injection device according to any one of the preceding claims, wherein during assembly of the injection device, the drive spring is preloaded such that prior to a first dose setting operation, the drive spring has enough energy to supply the force required to perform at least one dose delivery, optionally a plurality of dose deliveries, without any input of energy by the user into the drive spring.

21. An injection device according to any one of the preceding claims, wherein during assembly of the injection device, the drive spring is preloaded such that prior to a first dose setting operation, the drive spring has enough energy to supply the force required to deliver the total deliverable volume of substance in the cartridge or syringe over one or more dose deliveries without the user needing to input any energy into the drive spring.

22. An injection device according to any one of the preceding claims, wherein the drive shaft comprises a first drive shaft portion and a separate second drive shaft portion, wherein:
the drive spring is configured to directly or indirectly urge the first drive shaft portion to rotate relative to the housing;
the first drive shaft portion is engaged with the second drive shaft portion such that the first drive shaft portion is rotationally fixed relative to the second drive shaft portion; and
the stop member is engaged between the dose setting member and the second drive shaft portion.

23. An injection device according to claim 22 when dependent on claim 7 or when dependent any claim dependent on claim 7, wherein:
the clutch member is integrally formed with or is attached to the second drive shaft portion such that the clutch member is axially fixed relative to the second drive shaft portion;
the second drive shaft portion is axially movable relative to the first drive shaft portion; and
when the clutch member is in its engaged position, the clutch member is engaged between the first drive shaft portion and the housing to prevent relative rotation between the first drive shaft portion and the housing.

24. A method of assembling an injection device, the injection device comprising:
a drive shaft comprising a threaded portion;
a plunger comprising a corresponding threaded portion for engaging with the threaded portion of the drive shaft; and
a compression spring;
the method comprising the steps of:
engaging a first end of the compression spring with the drive shaft;
engaging a second end of the compression spring with the plunger, such that the compression spring is engaged between the drive shaft and the plunger in the axial direction; and
rotating the plunger relative to the drive shaft to threadedly engage the plunger with the drive shaft and cause the plunger to move axially relative to the drive shaft, such that the compression spring is simultaneously compressed and torsioned between the plunger and the drive shaft.

25. An injection device for delivering a dose from a cartridge or syringe, the injection device comprising:
a housing for receiving the cartridge or syringe;
a drive shaft rotatably mounted within the housing;
a plunger threadedly engaged with the drive shaft; and
a compression spring axially engaged between the drive shaft and the plunger and configured to urge the plunger to move axially relative to the drive shaft and the housing for delivering a dose from the cartridge or syringe, in use;
wherein the compression spring is in a compressed and torsioned state.
